(19) United States
(12) Patent Application Publication
(10) Pub. No.: US 2006/0107732 A1
(43) Pub. Date: May 25, 2006

(54) Method and system for the detection and recording of adhesive deposits or components in paper and woodpulp production

(76) Inventor: Alexander Frank, Strasshof (AT)

Correspondence Address: JACOBSON HOLMAN PLLC, 400 SEVENTH STREET N.W., SUITE 600, WASHINGTON, DC, 20004, US

(21) Appl. No.: 11/327,355

(22) Filed: Jan. 9, 2006

Related U.S. Application Data
(63) Continuation of application No. PCT/AT04/00241, filed on Jul. 6, 2004.

(30) Foreign Application Priority Data
Jul. 8, 2003 (AT) GM 482/2003

(51) Int. Cl. G01N 33/34

(52) U.S. Cl. 73/53.03; 162/198; 162/263

(57) ABSTRACT

The invention relates to a method and system for the detection and recording of adhesive deposits or components in the production cycle for paper and woodpulp production or paper and woodpulp processing, whereby a body (6), in particular, a rotationally symmetrical body, is partly dipped in aqueous suspension (2) from the paper and woodpulp production, or paper and woodpulp processing and a flotation of particles contained in the suspension is brought about, by introduction of at least one gas into the suspension (2) which does not react with said suspension (2). After removal of the body (6) from the suspension, material deposited on the body, in particular, in the region of the surfaces of the suspension, is visualised with an indicator in an analytical unit (10), whereby a simple and economical detection of said adhesive components or deposits can be achieved.

This is a continuation of PCT/AT04/000241 filed Jul. 6, 2004 and published in German.

### BACKGROUND OF THE INVENTION

The present invention relates to a method and a system for the detection and recording of adhesive deposits or adhesive components in the production cycle for paper and woodpulp production or paper and woodpulp processing, as well as the use of such a method and/or system.

### Prior Art

In the context of paper and pulp production, or paper and pulp processing, it is known that adhesive or sticky deposits and/or components may occur or form at various points in the production cycle, which may affect the production process and/or lead to minor-quality products. Such adhesive or sticky deposits or components are partially referred to as “stickies”, wherein such deposits or components may occur during the processing of both primary and secondary fibres. They may, for instance, comprise resins contained in the raw materials used in paper and pulp production, or such sticky deposits or components may be introduced into the production process when processing waste paper.

In order to determine the presence of such adhesive deposits or components in a production cycle, it is, for instance, known to take samples at different points of the production cycle and, for instance, subject the same to complex and expensive primarily physical separation methods in order to examine stock flows for the presence of such adhesive deposits or components, as may, for instance, be taken from U.S. Pat. No. 6,010,593.

### SUMMARY OF THE INVENTION

The present invention aims to provide a method and a system for the detection and recording of adhesive deposits or adhesive components in the production cycle for paper and woodpulp production or paper and woodpulp processing, wherein it is feasible by simple, quick and cost-effective procedures, and simple devices, to quickly detect, at different points of such a production cycle, the presence of adhesive deposits or components and, if necessary, take counter measures required to maintain a proper production course.

To solve this object, the method according to the invention of the initially described kind is essentially characterized in that a body and, in particular, a rotationally symmetrical body is partly dipped into an aqueous suspension from paper and woodpulp production or paper and woodpulp processing, that a flotation of particles contained in the suspension is brought about by the introduction into the suspension to be examined, of at least one gas that does not react with said suspension, and that, after the removal of the body from the suspension, substances deposited on the body, particularly in the region of the surface of said suspension, are visualised by the aid of an indicator. Due to the fact that, according to the invention, a body is partly dipped into an aqueous suspension and a flotation is brought about by the introduction or injection of at least one gas into said aqueous suspension, it is feasible by simple steps to detect the presence of adhesive deposits or components on the body dipped into the suspension and, in particular, formed by a rotationally symmetrical body, whereupon an evaluation of the adhesive deposits is carried out using an indicator. By the application of the method according to the invention it is, thus, feasible to renounce expensive and complex, particularly physical separation methods and simply carry out a determination of optionally present deposits after having taken samples at different points and upon preparation of an aqueous suspension. By introducing at least one gas that does not react with the suspension to be examined, a flotation is caused such that adhesive deposits or components possibly contained in the suspension to be examined will, in particular, collect in the region of the surface of the suspension, or boundary layer between the suspension and the superimposed atmosphere, and accordingly adhere to the surface of the body partly dipped into the suspension. After having removed the body from the suspension, it is subsequently readily feasible to detect, by the aid of an indicator, adhesive deposits or components deposited on the body, particularly in the region of the interface, during the immersion of the body, whereupon optionally required counter measures may be taken in a simple manner in order to maintain the proper course of production.

To further enhance the thorough mixing of the suspension and, hence, improve the evaluation of adhesive deposits or components possibly contained in the suspension, it is proposed according to another preferred embodiment that stirring of the suspension to be examined is additionally effected. Such stirring may, for instance, be effected by the aid of a magnetic stirrer or series stirrer.

For the safe evaluation and reliable collection of the adhesive deposits or components possibly contained in the suspension to be examined in order to ensure a proper detection of the same, it is proposed according to a further preferred embodiment that flotation is maintained over a period of 20 minutes to 3 hours and, in particular, 1 hour so as to enable reliable statements to be made at a comparatively short and simple process control.

As pointed out above, the method according to the invention renders feasible the examination of stock flows at different points of the production course, such stock flows in the context of paper and pulp production partially having strongly varying consistencies and densities. In order to enable the safe detection of possibly contained adhesive deposits or components by the flotation proposed according to the invention, it is contemplated according to a further preferred embodiment that an approximately 0.5 to 5% aqueous suspension is used as the suspension to be examined. A dilution of the suspension to be examined to the indicated range, which is to be carried out, if necessary, will enable the proper and reliable realization of the flotation proposed according to the invention.

In order to avoid any adverse effects on the evaluation procedure by materials or components of the suspension to be examined possibly additionally present on the outer surface of the immersed body, it is proposed according to a preferred embodiment that the body is cleaned by rinsing with water prior to visualizing the deposited, adhering substances. By such cleaning, particularly with water, all of the materials additionally present on the surface of the body dipped into the suspension will be removed, while only the adhesive deposits or components that are of interest for an evaluation will remain on the body surface.

As a function of the indicator used in the context of the evaluation, further treatment of the adhesive components adhering to the body may be required, wherein, in this respect, it is contemplated according to a further preferred embodiment that the body is dried at temperatures of between 40 and 75° C. prior to being treated or contacted by an indicator. Such drying, for instance, in a drying cabinet or kiln enhances the adherence of the adhesive components to the outer surface of the body dipped into the suspension to be examined, thus facilitating the subsequent evaluation procedure.

For a particularly cost-effective and time-saving evaluation, it is contemplated according to another preferred embodiment that the visualisation of the deposited substances is carried out by dipping the body into an aqueous indicator solution. Different indicator solutions are known and available, wherein, in the context of the adhesive deposits or components to be detected and recorded in accordance with the invention, it is further contemplated that the indicator solution contains a mixture of red dyes and, in particular, methyl red and neutral red or safranin O at a concentration of 0.05 to 0.5% and, in particular, 0.8 to 0.15%, as in correspondence with a further preferred embodiment of the method according to the invention.

A particularly simple a quick evaluation will preferably be effected in that, after the visualisation of the deposits, the latter are compared with an evaluation scale. In addition, or as an alternative, photos may, for instance, be taken after the visualisation of the deposits, particularly for documentation purposes, or an optionally at least partly automatic evaluation and quantification by the aid of digital photography and electronic image evaluation may be provided.

For a particularly simple process control using simple materials, it is proposed according to a further preferred embodiment that a plastic tube open on both ends is used as said rotationally symmetrical body. Such a plastic tube can be adapted to the substances to be detected, particularly in terms of its surface nature, wherein, in this respect, it is proposed according to a further preferred embodiment that the body is made of hydrophobic material.

In order to obtain a reliable flotation effect likewise by using simple materials and devices at least in the region of the body to be dipped into the suspension, it is proposed according to a further preferred embodiment that the introduction of gases into the suspension to be examined is effected by inserting a glass frit into the body to be introduced into the suspension and passing gas through said glass frit.

To achieve the objects set out in the beginning, a system for the detection and recording of adhesive deposits or adhesive components in the production cycle for paper and woodpulp production or paper and woodpulp processing is, moreover, provided according to the invention, which system comprises:
a receptacle for a suspension to be examined, which is derived from paper and woodpulp production or paper and woodpulp processing,
a body capable of being introduced into said suspension and, in particular, a rotationally symmetrical body,
a feed line for introducing into said suspension at least one gas that does not react with the suspension to be examined, whereby a flotation of particles contained in the suspension is brought about, and an evaluation device comprising at least one indicator for contacting said body and substances adhering thereto or deposited thereon.

As already pointed out above, it is, thus, feasible by simple means to provide a reliable evaluation and reliable detection of adhesive deposits or components possibly contained in a suspension taken from a production cycle for paper and woodpulp production. As already pointed out as well, it is feasible by the system according to the invention to readily take a sample of a suspension to be examined each at the most different points of the production cycle, with the sample being introduced into the receptacle, whereupon, after the formation of a flotation by the introduction of gas into the suspension to be examined as well as the immersion of a body capable of being introduced into the suspension, a simple evaluation device will subsequently do.

For a body to be dipped into the suspension to be examined, which can be produced in a particularly cost-effective manner and adapted to optionally different purposes of use, it is proposed according to a preferred embodiment that a plastic tube open on both ends is used as said rotationally symmetrical body.

In order to ensure the reliable introduction of gas into the suspension to be examined to readily bring about the flotation effect, it is proposed according to a preferred embodiment that a glass frit is inserted in the body to be introduced into the suspension, which gas frit is connected with the gas feed line.

With a view to ensuring the safe adherence of adhesive deposits or components to be detected and recorded, it is, moreover, contemplated that the body is made of hydrophobic material as in correspondence with a further preferred embodiment of the system according to the invention.

In order to ensure proper mixing of the suspension and, hence, an improved evaluation, it is proposed according to a preferred embodiment that a stirring means and, in particular, a series stirrer is additionally provided.

For a homogenized adherence of the particles to be deposited in the region of the surface of the suspension, it is proposed that the rotationally symmetrical body is drivable and coupleable to a drive of the stirring means, as in correspondence with a further preferred embodiment of the invention.

For a simple evaluation it is, moreover, proposed in a preferred manner that the evaluation device comprises an evaluation scale.

As already indicated above, additional ingredients of the suspension to be examined, besides the adhesive deposits or components to be detected, may attach to the body to be dipped into the suspension and, in particular, rotationally symmetrical body, which additional ingredients have to be removed, in a suitable manner, prior to said evaluation. In this context, it is proposed according to further preferred embodiment that a rinsing device is additionally provided.

For the preparation of said evaluation, optionally as a function of the indicator employed, it is proposed according to another preferred embodiment that a drying kiln is additionally provided.

In order to maintain constant examination conditions, it is proposed according to a further preferred embodiment that a heating means for tempering the suspension to be examined is additionally provided.

The method according to the invention as well as the system according to the invention are suitable, in particular, for use in the detection and recording of adhesive deposits or components occurring in the production cycle for paper and woodpulp production or paper and woodpulp processing, in particular, resins, adhesives, hot-melt adhesives, binders, residues from waste paper or the like.

### SHORT DESCRIPTION OF THE DRAWINGS

In the following, the invention will be explained in more detail by way of exemplary embodiments schematically illustrated in the accompanying drawing. Therein:

FIG. 1 is a schematic flow chart for the implementation of the method according to the invention;

FIG. 2 is a schematic partial view of the system according to the invention, for carrying out the method according to the invention, with the evaluation device of the system according to the invention having been omitted din FIG. 2; and

FIG. 3 is a schematic block diagram depicting components of the system according to the invention.

### DESCRIPTION OF THE PREFERRED EMBODIMENTS

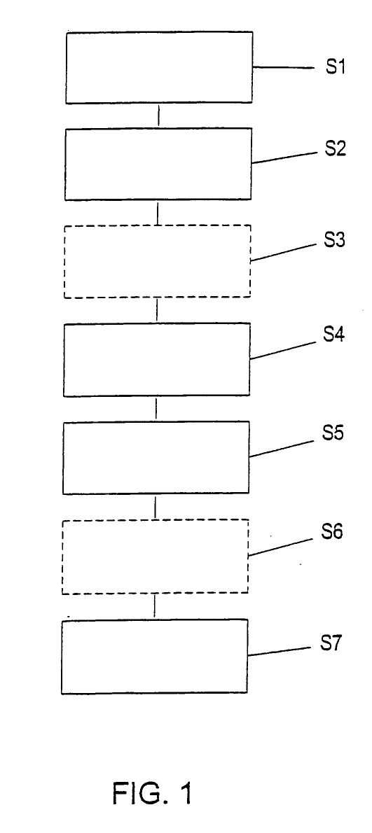

In the flow chart represented in FIG. 1, step S1 indicates the removal of a suspension to be examined, from the production cycle for paper and woodpulp production.

In step S2, a flotation is brought about by the introduction of a gas that does not react with the suspension to be examined, while a body and, in particular, a rotationally symmetrical body is, at the same time, dipped into the suspension to be examined.

S3 additionally indicates that stirring is optionally effected using, for instance, a magnetic stirrer to obtain a better distribution of the particles contained in the suspension so as to ensure the proper deposition of adhesive deposits or components possibly contained in the suspension.

The flotation is, for instance, maintained over a period of approximately 1 hour.

In step S4, the removal of the body dipped into the suspension, from the suspension to be examined takes place, whereupon, in step S5, rinsing of the body, for instance, with water is effected in order to remove components or materials additionally adhering to the body dipped into the suspension besides the adhesive deposits or components deposited particularly in the region of the boundary layer, between the suspension to be examined and the atmosphere.

In step S6, drying of the body is indicated, which is to be additionally performed in an optional manner.

After this, step S7 comprises the evaluation of the adhesive deposits or components deposited on the surface of the body, using an indicator solution.

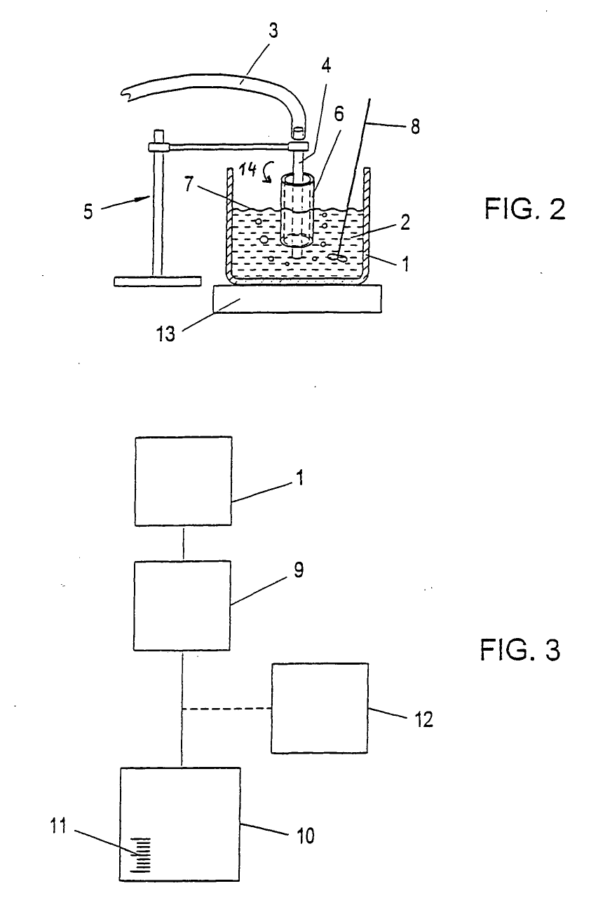

In FIG. 2, a receptacle containing a suspension 2 to be examined is schematically denoted by 1. Via a feed line 3, a gas is introduced into the suspension to be examined, through a glass frit 4 dipped into the suspension to be examined, to cause the formation of a flotation. A retaining means for the glass frit 4 is indicated at 5.

A particularly rotationally symmetrical body, for instance a plastic tube 6 formed with a hydrophobic surface, is dipped into the suspension in a manner substantially concentric with the glass frit 4, whereby adhesive deposits or components possibly present in the suspension to be examined will collect in the region of the boundary layer denoted by 7, by attaching to the surface of the body 6 comprised of a plastic tube formed with a hydrophobic surface.

To further enhance the mixing of the suspension 2 to be examined, a stirring means 8, for instance a magnetic stirrer or series stirrer, is additionally indicated.

In order to homogenize the adherence to the rotationally symmetrical body 6, it may further be provided that the body 6 is likewise rotatable in the sense of arrow 14, wherein a driving means (not illustrated) for the stirrer 8 is coupleable to a driving means for the body 6, or a common driving means is provided.

After having left the plastic tube 6 for a sufficiently long period of flotation, e.g. 1 hour, in the suspension to be examined, the body 6 is removed from the suspension to be examined and introduced into a rinsing device, which is denoted by 9 in FIG. 3, FIG. 3 indicating the receptacle 1 with the accessory means additionally shown in FIG. 2 to precede the rinsing device 9.

In the rinsing device 9, the removal of materials possibly additionally adhering to the surface is effected, whereupon the plastic tube 6 dipped into the suspension is conveyed from the rinsing device 9 to an evaluation device 10. In this evaluation device, the plastic tube is, for instance, dipped into an appropriate indicator solution, followed by an evaluation using an evaluation or assessment scale 11 as reference.

If desired, the plastic tube, prior to being introduced into the evaluation device 10, may be dried in a drying kiln 12 along with the attachments adhering to its surface in order to facilitate or improve the evaluation to follow. In order to maintain constant examination conditions, a heating means 13 is, moreover, indicated in FIG. 2 below the receptacle 1 to appropriately adjust the temperature of the suspension 2 to be examined.

1-29. (canceled)

30. A method detecting and recording adhesive deposits or adhesive components in the production cycle for paper and woodpulp production or paper and woodpulp processing, wherein a body is partly dipped into an aqueous suspension from paper and woodpulp production or paper and woodpulp processing, that a flotation of particles contained in the suspension is brought about by the introduction into the suspension to be examined, of at least one gas that does not react with said suspension, and that, after the removal of the body from the suspension, substances deposited on the body are visualised by the aid of an indicator.

31. The method according to claim 30, wherein a rotationally symmetrical body is used.

32. The method according to claim 30, wherein substances deposited on the body in the region of the surface of the suspension are visualised by the aid of said indicator.

33. The method according to claim 30, wherein stirring of the suspension to be examined is additionally effected.

34. The method according to claim 30, wherein flotation is maintained over a period of 20 minutes to 3 hours.

35. The method according to claim 34, wherein flotation is maintained over a period of approximately 1 hour.

36. The method according to claim 30, wherein an approximately 0.5 to 5% aqueous suspension is used as the suspension to be examined.

37. The method according to claim 30, wherein the body is cleaned by rinsing with water prior to visualizing the deposited, adhering substances.

38. The method according to claim 30, wherein the body is dried at temperatures of between 40 and 75° C. prior to being treated or contacted by an indicator.

39. The method according to claim 30, wherein the visualisation of the deposited substances is carried out by dipping the body into an aqueous indicator solution.

40. The method according to claim 39, wherein the indicator solution contains a mixture of red dyes at a concentration of 0.05 to 0.5%.

41. The method according to claim 40, wherein a mixture of methyl red and neutral red or safranin O is used as said indicator solution.

42. The method according to claim 40, wherein the indicator solution contains said mixture of red dyes at a concentration of 0.8 to 0.15%.

43. The method according to claim 30, wherein, after the visualisation of the deposits, the latter are compared with an evaluation scale.

44. The method according to claim 30, wherein a plastic tube open on both ends is used as said rotationally symmetrical body.

45. The method according to claim 30, wherein the body is made of hydrophobic material.

46. The method according to claim 30, wherein the introduction of gases into the suspension to be examined is effected by inserting a glass frit into the body to be introduced into the suspension and passing gas through said glass frit.

47. A system for the detection and recording of adhesive deposits or components in the production cycle for paper and woodpulp production or paper and woodpulp processing, comprising:
a receptacle for a suspension to be examined, which is derived from paper and woodpulp production or paper and woodpulp processing,
a body which is capable of being introduced into said suspension,
a feed line for introducing into the suspension to be examined at least one gas that does not react with said suspension, whereby a flotation of particles contained in the suspension is brought about, and
an evaluation device comprising at least one indicator for contacting said body and substances adhering thereto or deposited thereon.

48. The system according to claim 47, wherein the body capable of being introduced into said suspension is formed by a rotationally symmetrical body.

49. The system according to claim 48, wherein a plastic tube open on both ends is used as said rotationally symmetrical body.

50. The system according to claim 47, wherein a glass frit is inserted in the body to be introduced into the suspension, which glass frit is connected with the gas feed line.

51. The system according to claim 47, wherein the body is made of hydrophobic material.

52. The system according to claim 47, wherein a stirring means and, in particular, a series stirrer is additionally provided.

53. The system according to claim 52, wherein the rotationally symmetrical body is drivable and coupleable to a drive of the stirring means.

54. The system according to claim 47, wherein the evaluation device comprises an evaluation scale.

55. The system according to claim 47, wherein a rinsing means is additionally provided.

56. The system according to claim 47, wherein a drying kiln (12) is additionally provided.

57. The system according to claim 47, wherein a heating means for tempering the suspension to be examined is additionally provided.

58. The use of the method according to claim 47 for the detection and recording of adhesive deposits or components occurring in the production cycle for paper and woodpulp production or paper and woodpulp processing, in particular, resins, adhesives, hot-melt adhesives, binders, residues from waste paper or the like.
